(19) United States
(12) Patent Application Publication
(10) Pub. No.: US 2024/0325588 A1
(43) Pub. Date: Oct. 3, 2024

(54) FILTER FOR AIR PURIFICATION AND MANUFACTURING METHOD THEREOF

(71) Applicants: HYUNDAI MOTOR COMPANY, SEOUL (KR); KIA CORPORATION, SEOUL (KR); UNIVERSITY OF SEOUL INDUSTRY COOPERATION FOUNDATION, SEOUL (KR)

(72) Inventors: Kwangmo Seong, Bucheon-si (KR); Jungsik Kim, Seoul (KR)

(21) Appl. No.: 18/425,413

(22) Filed: Jan. 29, 2024

(30) Foreign Application Priority Data
Mar. 30, 2023 (KR) 10-2023-0042080

(51) Int. Cl. A61L 9/20

(52) U.S. Cl. CPC A61L 9/205

(57) ABSTRACT

A filter for air purification includes a plurality of mesh slits, each mesh slit including a metal mesh support, a phosphor layer coated on a surface of the mesh metal support, a plurality of transition metal particles loaded on the phosphor layer, and a photocatalyst layer.

### CROSS REFERENCE TO RELATED APPLICATIONS

This application claims the benefit of priority to Korean Patent Application No. 10-2023-0042080, filed on Mar. 30, 2023 in the Korean Intellectual Property Office, the disclosure of which is incorporated herein by reference.

### TECHNICAL FIELD

The present disclosure relates to a filter for air purification using a mesh slit coated with a phosphor, a transition metal, and a photocatalyst and a method of manufacturing the filter for air purification.

### BACKGROUND

As air pollution is getting worse in recent years, demand for air purification systems (air purifies, air purification device, and the like) to improve the quality of air is increasing. As a result, various studies have been extensively conducted into methods for effectively purifying indoor air polluted by various air pollutants, vehicle exhaust gases, volatile organic compounds (VOCs), harmful gases, odors, viruses, and the like. Among these studies, air purifying technology by using a photocatalyst material having strong photocatalytic oxidation function has drawn considerable attention.

Titanium dioxide (TiO₂), known as a representative photocatalyst material, generates radicals with strong oxidizing power when exposed to ultraviolet light. Such radicals may decompose various environmental pollutants present in water or in the air into harmless carbon dioxide and water. In addition, because titanium dioxide is chemically very stable without changing even when exposed to light, it is advantageous to use titanium dioxide semi-permanently.

Meanwhile, active oxygen (O₂⁻) or hydroxyl radicals (·OH) generated by photoreaction have higher oxidizing power than conventional chlorine (Cl₂) or ozone (O₃), thereby having superior sterilization against harmful viruses and bacteria and deodorization against odors.

However, although titanium dioxide is an excellent photocatalyst as a single-component material, photocatalytic oxidation reaction occurs only upon absorbing ultraviolet light having a high energy (UV, λ≤390 nm) due to a large energy bandgap (e.g., a band gap of anatase phase: 3.2 eV). Therefore, in the case where titanium dioxide is exposed to sunlight, only about 3 to 4% of ultraviolet light contained in the sunlight may be absorbed by the titanium dioxide.

In addition, there are many limits to directly apply titanium dioxide itself to apparatuses for reducing air pollutants, and the like. Therefore, numerous studies are being conducted to effectively use photocatalyst materials in air purification apparatuses.

### SUMMARY

In general, a photocatalyst-containing material prepared in the form of a plurality of beads and inserted into a mesh frame is used to utilize photocatalyst materials in air purifiers. However, in the case where the plurality of beads are randomly inserted into a frame, frictional abrasion is caused by contact between the beads in a vibrating environment, for example, in a vehicle. Due to such frictional abrasion, beads may be damaged or the photocatalyst is detached from the surfaces of the beads to generate fine powder (containing Cu, Sr, Ti, or the like) and the fine powder is adhered to a surface of an evaporator core or a heater core causing a problem of inducing potential difference corrosion. Furthermore, in the case where the beads are continuously damaged, the amount of the photocatalyst in the filter decreases, resulting in deterioration of photocatalytic oxidation performance.

Therefore, an aspect of the present disclosure is to provide a filter for air purification having a structure capable of maximizing photocatalytic oxidation efficiency and minimizing damage of a photocatalyst-containing material and a manufacturing method thereof.

However, the technical problems to be solved by the present disclosure are not limited to the aforementioned problems, and any other technical problems not mentioned herein will be clearly understood from the following description by those skilled in the art to which the present disclosure pertains.

Additional aspects of the disclosure will be set forth in part in the description which follows and, in part, will be obvious from the description, or may be learned by practice of the disclosure.

In accordance with an aspect of the disclosure, a filter for air purification including a plurality of mesh slits stacked, wherein each of the mesh slits includes a metal mesh support, a phosphor layer coated on a surface of the mesh metal support, a plurality of transition metal particles supported on the phosphor layer, and a photocatalyst layer coated on the phosphor layer on which the plurality of transition metal particles are supported.

In addition, a rubber packing may be disposed between the plurality of mesh slits.

In addition, the phosphor layer may include a phosphor material, a binder, and zeolite.

In addition, the phosphor material may include at least one selected from CaAl₂O₄:(Eu,Nd)-based, SrAl₂O₄:(Eu,Dy)-based, Sr₄Al₁₄O₂₅:(Eu,Dy)-based, BaAl₂O₄:(Eu,Dy)-based, (Sr,Ba)₂MgSi₂O₇:(Eu,Dy)-based, Ba₄(Si₃O₈)₂:(Eu,Dy)-based, and [Ca,Sr,Ba]—Al—O-based compounds.

In addition, the binder may include at least one selected from sodium silicate (Na₂SiO₃), sodium polyphosphate (NaPO₃)ₙ, liquid silica, and glaze.

In addition, the transition metal particles may include at least one selected from Cr, Mn, Fe, Co, Ni, Cu, Zn, Zr, Ru, Rh, Pd, Ag, Ta, W, Pt, and Au.

In addition, the photocatalyst layer may include at least one photocatalyst material selected from titanium dioxide (TiO₂), graphite carbon nitride (g-C₃N₄), and a combination thereof.

In addition, the photocatalyst layer may further include SiO₂.

In accordance with another aspect of the disclosure, a method of manufacturing a filter for air purification includes preparing a plurality of metal mesh support, obtaining a phosphor-coated support by coating a surface of each support with a phosphor layer, supporting a plurality of transition metal particles on the phosphor-coated support, coating a photocatalyst layer on the phosphor-coated support on which the transition metal particles are supported to obtain a plurality of mesh slits, and stacking the plurality of mesh slits.

In addition, the obtaining of a phosphor-coated support by coating a surface of a support with a phosphor layer may include preparing a phosphor slurry by mixing phosphor powder, a binder, zeolite, and distilled water, and dipping the support in the phosphor slurry and drying and heat-treating a resultant.

In addition, the phosphor powder may include at least one phosphor material selected from CaAl₂O₄:(Eu,Nd)-based, SrAl₂O₄:(Eu,Dy)-based, Sr₄Al₁₄O₂₅:(Eu,Dy)-based, BaAl₂O₄:(Eu,Dy)-based, (Sr,Ba)₂MgSi₂O₇:(Eu,Dy)-based, Ba₄(Si₃O₈)₂:(Eu,Dy)-based, and [Ca,Sr,Ba]—Al—O-based compounds, and the binder may include at least one selected from sodium silicate (Na₂SiO₃), sodium polyphosphate (NaPO₃)ₙ, liquid silica, and glaze.

In addition, the loading of the plurality of transition metal particles on the phosphor-coated support may include preparing a transition metal loading solution by dissolving a transition metal salt in an alcohol, dipping the phosphor-coated support in the transition metal loading solution, followed by sonication, and drying and heat-treating a resultant.

In addition, the transition metal salt may include at least one selected from copper nitrate trihydrate [Cu(NO₃)₂·3H₂O] and copper sulfate pentahydrate (CuSO₄·5H₂O).

In addition, the coating of the photocatalyst layer may be performed by applying at least one method selected from a sol-gel method, a hydrothermal synthesis method, and a chemical vapor deposition (CVD) method thereto.

In addition, the coating of a photocatalyst layer on the phosphor-coated support on which the transition metal particles are loaded to obtain a plurality of mesh slits may include preparing a photocatalyst sol by mixing a photocatalyst precursor, an alcohol-based solution, and an acid performing hydrothermal synthesis on the phosphor-coated support on which the transition metal particles are loaded and the photocatalyst sol, and drying and heat-treating a resultant.

In addition, the photocatalyst precursor may include at least one selected from titanium tetra-isopropoxide [Ti(OCH(CH₃)₂)₄], tetrabutyl titanate [Ti(C₄H₉O)₄], and tetraethoxy titanium [Ti(OCH₂CH₃)₄].

In addition, the stacking of the plurality of mesh slits may further includes inserting a rubber packing between the mesh slits.

### BRIEF DESCRIPTION OF THE FIGURES

These and/or other aspects of the disclosure will become apparent and more readily appreciated from the following description of the embodiments, taken in conjunction with the accompanying drawings of which:

FIG. 1 is a cross-sectional view of a mesh slit of a filter for air purification according to an embodiment.

FIG. 2 is an exploded perspective view of a filter for air purification according to an embodiment.

FIG. 3A is a photograph of a sample of a filter for air purification prepared according to an embodiment.

FIG. 3B is a photograph of a filter for air purification prepared according to an embodiment after a vibration durability test (3.5 months).

FIG. 4A is a photograph of a sample of a filter for air purification prepared according to a comparative example.

FIG. 4B is a photograph of a filter for air purification prepared according to a comparative example after a vibration durability test (3.5 months).

### DETAILED DESCRIPTION

Various embodiments and terms used herein are not intended to limit technical features disclosed herein to the particular embodiments. However, it should be understood that it is not intended to limit the present disclosure to the particular forms disclosed, but on the contrary, the present disclosure is to cover all modifications, equivalents, and alternative falling within the spirit and the scope of the disclosure.

With respect to descriptions of the drawings, like reference numerals denote like elements.

Singular forms corresponding to items may include one item or a plurality of items unless the context clearly indicates otherwise.

Throughout the specification, the phrases “A or B”, “at least one of A and B”, “at least one of A or B”, “A, B or C”, “at least one of A, B and C”, and “at least one of A, B, or C” mean any one of the items listed together with the phrases or all possible combinations thereof.

The term “and/or” includes any combination of a plurality of related elements or any one of a plurality of related elements.

The terms “first”, “second”, and the like may be used to distinguish one element from another, without limiting the elements in other aspects (e.g., importance or order).

In addition, the terms ‘front’, ‘rear’, ‘upper’, ‘lower’, ‘side’, ‘left’, ‘right’, ‘top’, and ‘bottom’ used herein are defined based on the drawings, and shapes and positions of the components are not limited by these terms.

Also, it is to be understood that the terms such as “include”, “have”, or the like, are intended to indicate the existence of the features, numbers, operations, components, parts, or combinations thereof disclosed in the specification, and are not intended to preclude the possibility that one or more other features, numbers, operations, components, parts, or combinations thereof may exist or may be added.

When an element is “connected to”, “coupled to”, “supported by” or “brought into contact with” another element, it may be directly connected to, coupled to, supported by, or brought into contact with the other element or indirectly connected to, coupled to, supported by, or brought into contact with the other element via a third intervening element.

When an element is referred to as being “on” another element, it may be directly on the other element or intervening elements may be present.

The terms “about”, “substantially”, etc. used throughout the specification means that when a natural manufacturing and a substance allowable error are suggested, such an allowable error corresponds the value or is similar to the value, and such values are intended for the sake of clear understanding of the present disclosure or to prevent an unconscious infringer from illegally using the disclosure.

Hereinafter, filters for air purification and manufacturing methods thereof according to various embodiments will be described in detail with reference to the accompanying drawings.

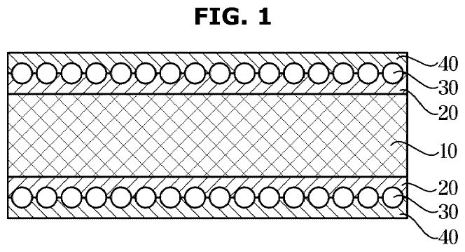

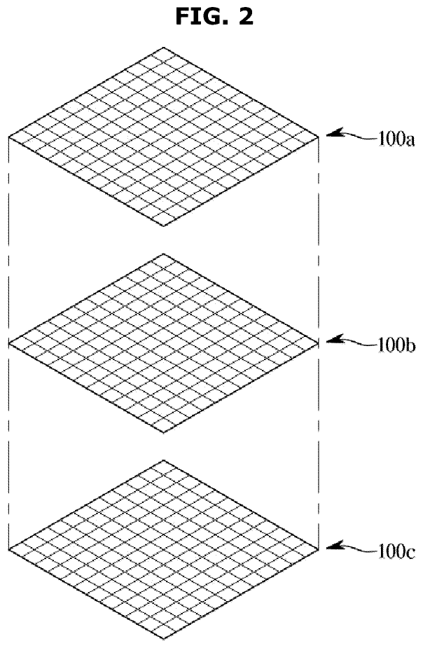

FIG. 1 is a cross-sectional view of a mesh slit of a filter for air purification according to an embodiment. FIG. 2 is an exploded perspective view of a filter for air purification according to an embodiment.

As shown in FIGS. 1 and 2, a filter for air purification according to an embodiment includes a stack structure of a plurality of mesh slits 100a, 100b, and 100c, wherein the mesh slit includes a metal mesh support 10, a phosphor layer 20, a plurality of transition metal particles 30, and a photocatalyst layer 40.

Referring to FIG. 1, a mesh slit according to an embodiment includes a metal mesh support 10, a phosphor layer 20 coated on the surface of the mesh metal support 10, a plurality of transition metal particles 30 loaded on the phosphor layer 20, and a photocatalyst layer 40 coated on the phosphor layer 20 on which the plurality of transition metal particles 30 are loaded.

The metal mesh support 10 may be formed of any metallic material well known in the art without limitation and may have a mesh structure to obtain superior air-permeability.

The phosphor layer 20 is coated on the surface of the mesh metal support 10. The phosphor layer 20 enables photocatalytic oxidation reaction of a photocatalyst even in a dark environment due to a self-emitting phosphor and allows nano-sized photocatalyst materials to be effectively attached to the support. Specifically, via hybridization of the self-emitting phosphor material and the photocatalyst, photocatalytic oxidation reaction of the photocatalyst may continue for a certain period of time by self-emitting property of the phosphor even after exposure to light is stopped.

The phosphor layer 20 include a phosphor material, a binder, and zeolite.

The phosphor material is a light-emitting material in the form of powder having high luminance and long phosphorescence, and any materials that absorb light and emit light may be used without limitation. The phosphor material emits light via excitation and de-excitation occurring by light such as sunlight received from the outside, and the photocatalyst material on the surface is photo-activated by the light emitted from the phosphor material.

Examples of the phosphor material may include at least one phosphor selected from CaAl₂O₄:(Eu,Nd)-based, SrAl₂O₄:(Eu,Dy)-based, Sr₄Al₁₄O₂₅:(Eu,Dy)-based, BaAl₂O₄:(Eu,Dy)-based (Sr,Ba)₂MgSi₂O₇:(Eu,Dy)-based, Ba₄(Si₃O₈)₂:(Eu,Dy)-based, and [Ca,Sr,Ba]—Al—O-based compounds. More specifically, the phosphor material may be CaAl₂O₄:(Eu²⁺, Nd³⁺) exhibiting blue fluorescence, SrAl₂O₄:(Eu²⁺, Nd³⁺) exhibiting green fluorescence, BaAl₂O₄:(Eu²⁺, Nd³⁺) exhibiting blue fluorescence, and Sr₄Al₁₄O₂₅:(Eu²⁺, Nd³⁺) exhibiting bluish green fluorescence, or the like.

The binder may include at least one inorganic material such as sodium silicate (Na₂SiO₃), sodium polyphosphate (NaPO₃)ₙ, liquid silica, and glaze.

The zeolite may be a raw material in a powder form represented by formula (WₘZₙO₂)ₙ·nH₂O (where W is Na, Ca, Ba, or Sr, and Z=Si+Al).

The plurality of transition metal particles 30 are loaded on the phosphor layer 20. A transition metal binds to a photocatalyst material to lower photo-activation energy of the photocatalyst material, and thus reactive radicals may be easily generated thereby not only under ultraviolet light but also under visible light.

The transition metal particles 30 serve to improve photocatalytic oxidation performance by lowering photo-activation energy of the photocatalyst. Specifically, the transition metal particles bind to the photocatalyst material to lower the photo-activation energy of the photocatalyst material so as to easily generate reactive radicals. During this process, additional photocatalytic oxidation reaction of pollutants may occur, and photocatalytic oxidation reaction efficiency may be significantly increased by such additional photocatalytic oxidation reaction compared to conventional materials including only a photocatalyst.

For example, the transition metal particles 30 may include at least one transition metal selected from Cr, Mn, Fe, Co, Ni, Cu, Zn, Zr, Ru, Rh, Pd, Ag, Ta, W, Pt, and Au and Cu is the most preferable.

The photocatalyst layer 40 is coated on the phosphor layer 20 on which a plurality of transition metal particles 30 are loaded. The photocatalyst layer 40 may be formed of a photocatalyst material in the form of a plurality of nanoparticles.

The photocatalyst material on the surface is photo-activated by external ultraviolet or visible light to generate highly reactive radicals (such as hydroxyl radicals and reactive oxygen species) capable of decomposing pollutants such as harmful gases and organic materials in the air.

The photocatalyst material may include titanium dioxide (TiO₂), graphite carbon nitride (g-C₃N₄), a hybrid material of these materials [g-C₃N₄/TiO₂:(Fe,Cu,Co,Ni,N)], or the like which have the ability to photodegrade various types of organic materials and harmful gases by photo-activation upon absorbing light such as sunlight. For example, in the case of titanium dioxide is used as the photocatalyst material, the photocatalyst material may be crystallized in an anatase phase.

The photocatalyst layer 40 may further include SiO₂ to increase binding strength with the phosphor layer 20 on which the plurality of transition metal particles 30 are loaded. By increasing the binding strength between the phosphor layer 20 on which the transition metal particles 30 are loaded and the photocatalyst layer 40, photocatalytic oxidation efficiency may further be increased.

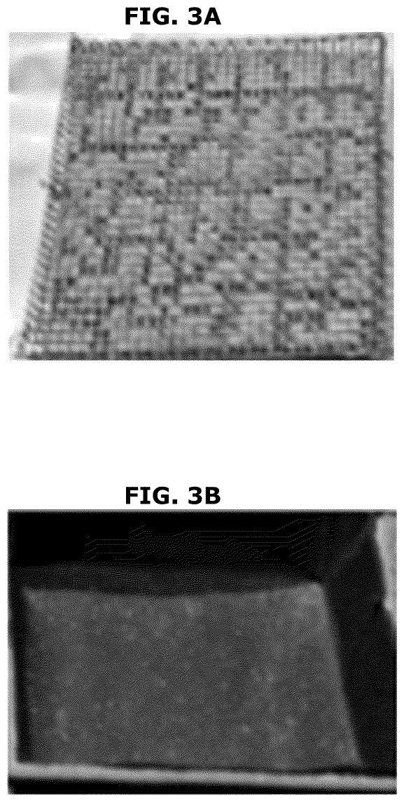

As shown in FIG. 2, the filter for air purification according to an embodiment includes a stack structure of a plurality of mesh slits 100a, 100b, and 100c. Although 3 mesh slits 100a, 100b, and 100c are shown in FIG. 3, the number thereof may be appropriately adjusted in required.

The plurality of mesh slits 100a, 100b, and 100c may be stacked to be spaced apart from each other not in contact with each other. For example, in order to prevent contact between the plurality of mesh slits, rubber packing (not shown) may be disposed between the plurality of mesh slits 100a, 100b, and 100c. Thus, friction between the mesh slits may be minimized even in a vibrating environment, thereby preventing generation of fine powder and deterioration in photocatalytic oxidation performance.

The number of the mesh slits may be appropriately adjusted if required. As the number of the stacked mesh slits increases, an amount of the photocatalyst increases, thereby increasing photocatalytic oxidation efficiency.

In an embodiment, the filter for air purification may further include an air-permeable body frame (not shown) to fix the plurality of mesh slits.

The body frame may be formed of any metallic material well known in the art without limitation and may have a mesh structure to obtain superior air-permeability.

A method of manufacturing a filter for air purification according to an embodiment includes preparing a plurality of metal mesh supports 10, obtaining a phosphor-coated support by coating a surface of each support with a phosphor layer 20, loading a plurality of transition metal particles 30 on the phosphor-coated support, coating a photocatalyst layer 40 on the phosphor-coated support on which the transition metal particles are loaded to obtain a plurality of mesh slits 100a, 100b, and 100c, and stacking the plurality of mesh slits 100a, 100b, and 100c.

First, in order to prepare a filter for air purification according to an embodiment, a plurality of metal mesh supports 10 are prepared.

The metal mesh support 10 may be formed of any metallic material well known in the art without limitation and may have a mesh structure to obtain superior air-permeability.

Subsequently, the surface of the support 10 is coated with the phosphor layer 20 to obtain the phosphor-coated support.

In an embodiment, for coating of the phosphor layer 20, phosphor powder, a binder, zeolite, and distilled water are mixed to prepare a phosphor slurry. The support is dipped in the phosphor slurry, and then dried and heat-treated.

The phosphor powder may be a CaAl₂O₄:(Eu,Nd)-based, SrAl₂O₄:(Eu,Dy)-based, Sr₄Al₁₄O₂₅:(Eu,Dy)-based, BaAl₂O₄:(Eu,Dy)-based, (Sr,Ba)₂MgSi₂O₇:(Eu,Dy)-based, Ba₄(Si₃O₈)₂:(Eu,Dy)-based, or [Ca,Sr,Ba]—Al—O-based compound.

The binder may be sodium silicate (Na₂SiO₃), glaze (glaze), or the like, but is not limited thereto.

The zeolite may be a raw material in a powder form represented by formula (WₘZₙO₂)ₙ·nH₂O (where W is Na, Ca, Ba, or Sr, and Z=Si+Al).

For example, the phosphor support may be prepared as follows. Phosphor powder (Sr₄Al₁₄O₂₅:Eu²⁺,Dy³⁺, etc.) and a mixture of a binder (Na₂SiO₃) and zeolite powder were weighed in a weight ratio of 1:0.5 to 1 and then mixed with distilled water by stirring to prepare a slurry. The support is dipped in the phosphor slurry to coat the support with a semi-solid phosphor layer and then dried and heat-treated at room temperature and in an electric oven to form a hard phosphor layer.

In the case where the phosphor is prepared as beads without a support, a problem of bead bursting may occur during a heat treatment process unless moisture is removed by appropriately controlling temperature, and thus a sufficient drying time is required to remove moisture. However, in the case of using the mesh slit as a support, the beads are sufficiently dried at phosphor at 100° C. within several dozens of minutes (≤20 min) by adjusting a thickness of the coating layer within several dozens of to several hundreds of micrometers. Meanwhile, although

Despite the above descriptions, the method of manufacturing the phosphor slurry and the method of coating the phosphor layer are not limited to the above-described examples.

Subsequently, the plurality of transition metal particles 30 are loaded on the surface of the phosphor-coated support by using a transition metal mixed solution.

In an embodiment, in order to load the plurality of transition metal particles 30 on the surface of the phosphor-coated support, a transition metal loaded solution is prepared by dissolving a transition metal salt in an alcohol, and the phosphor-coated support is added to the transition metal loaded solution and sonicated and filtered, followed by drying and heat treatment.

In this case, the transition metal salt may include at least one selected from copper nitrate trihydrate [Cu(NO₃)₂·3H₂O] and coper sulfate pentahydrate (CuSO₄·5H₂O). In addition, a concentration of the transition metal support solution may be from 0.600 to 0.170 M, preferably from 0.620 to 0.165 M. When the concentration range described above is satisfied, the effect on increasing photocatalyst efficiency may be increased.

For example, copper nitrate trihydrate is dissolved in an alcohol to prepare a solution having a concentration of 0.62 M to 0.165 M. The phosphor-coated support is added to the solution, followed by sonication. Then, the resultant is dried and heat-treated at room temperature and in an electric oven, thereby preparing a phosphor-coated support on which the transition metal particles are loaded. Specifically, the dried phosphor-coated support on which the transition metal particles are loaded may be heat-treated in a reducing atmosphere by injecting a nitrogen-hydrogen mixed gas.

Subsequently, a photocatalyst sol is prepared and applied to the surface of the phosphor-coated support on which the transition metal particles are loaded to form the photocatalyst layer 40, followed by heat treatment to prepare a plurality of mesh slits.

In the case where the mesh support is not used, a filtering process is additionally required after loading the transition metal particles and/or coating the photocatalyst layer. However, in the case of using the mesh support, an additional filtering process is not required and thus the process may be simplified.

The photocatalyst precursor may be a titanium precursor. The titanium precursor may be, for example, titanium tetra-isopropoxide [Ti(OCH(CH₃)₂)₄], tetrabutyl titanate [Ti(C₄H₉O)₄], and tetraethoxy titanium [Ti(OCH₂CH₃)₄], and titanium tetra-isopropoxide may be the most preferable, without being limited thereto.

The photocatalyst layer may be coated by applying at least one method selected from a sol-gel method, a hydrothermal synthesis method, and a chemical vapor deposition (CVD) method.

In an embodiment, in order to coat the surface of the phosphor-coated support on which the transition metal particles are loaded with the photocatalyst layer 40, a photocatalyst precursor, an alcohol solution, and an acid are mixed to prepare a photocatalyst sol, and the photocatalyst layer is coated by hydrothermal synthesis of the phosphor-coated support on which the transition metal particles are loaded and the photocatalyst sol, followed by drying and heat treatment.

According to hydrothermal synthesis, for example, titanium tetra-isopropoxide (TTIP, Ti(OCH(CH₃)₂)₄) as a titanium precursor, hydrous ethanol, an acid solution (HNO₃), and distilled water are mixed and stirred to prepare a titanium sol solution. The phosphor-coated support on which the transition metal particles are loaded and the titanium sol solution are subjected to hydrothermal synthesis in an autoclave at a temperature of 110 to 200° C. to coat the titanium dioxide on the phosphor-coated support on which the transition metal particles are loaded. The reactor is naturally cooled at room temperature and dried to obtain a plurality of mesh slits.

For example, in the case of using titanium dioxide as the photocatalyst, the method may further include heat treatment at a temperature of 300 to 600° C. for 2 to 8 hours for crystallization of the coated titanium dioxide into an anatase phase.

In addition, the photocatalyst sol may further include a tetraethyl orthosilicate solution. By adding an appropriate amount of a tetra-ethyl orthosilicate (TEOS, Si(OC₂H₅)₄) solution together with the titanium precursor, binding strength (adhesiveness) between the phosphor and TiO₂ may be increased.

In addition, to prepare the filter for air purification according to an embodiment, the plurality of mesh slits 100a, 100b, and 100c prepared as described above are stacked. In this case, the mesh slits may be stacked to be spaced apart from each other at a predetermined interval not to be in contact with each other. For example, the method may further include inserting rubber packing between the mesh slits to prevent contact between the plurality of mesh slits 100a, 100b, and 100c in a process of stacking the plurality of mesh slits 100a, 100b, and 100c. Thus, friction between the photocatalyst-containing beads is minimized even in a vibrating environment to prevent generation of fine powder and deterioration in photocatalytic oxidation performance.

In addition, an air-permeable body frame (not shown) may further be added to fix the stacked mesh slits outside the plurality of mesh slits.

Hereinafter, the present disclosure will be described in more detail according to the following examples. However, the following examples are merely presented to exemplify the present disclosure, and the scope of the present disclosure is not limited thereto.

### EXAMPLES

### (1) Example: Preparation of Filter for Air Purification 1

Sr₄Al₁₄O₂₅:Eu²⁺,Dy³⁺ powder, as a phosphor, and a mixture of Na₂SiO₃, as a binder, and zeolite powder were weighed in a weight ratio of 1:0.5 to 1 and then mixed with distilled water by stirring to prepare a slurry. A metal mesh slit as a support was dipped in the phosphor slurry for dip coating. The mesh slit coated with a semi-solid phosphor slurry was dried at room temperature (at about 25° C., for 2 hours) and in an electric oven (at 100° C., for 20 min) and heat-treated in an electric furnace (at 350° C., for 2 hours) to prepare a hard Sr₄Al₁₄O₂₅:Eu²⁺,Dy³⁺-coated slit.

Subsequently, copper nitrate trihydrate was dissolved in ethanol and stirred at 70° C. for 1 hour to prepare a copper solution. The Sr₄Al₁₄O₂₅:Eu²⁺,Dy³⁺-coated slit prepared as described above was added to the copper solution, followed by sonication for 10 minutes. The resultant was dried at room temperature (at about 25° C., for 3 hours) and in an electric oven (at 100° C., for 2 hours) and heat-treated in an electric furnace (in a 90% N₂-10% H₂ reducing atmosphere, at 300° C., for 2 hours) to prepare a Cu—Sr₄Al₁₄O₂₅:Eu²⁺,Dy³⁺-coated slit.

Subsequently, titanium tetra-isopropoxide (TTIP, Ti(OCH(CH₃)₂)₄) as a titanium precursor, hydrous ethanol, an acid solution (HNO₃), and distilled water were mixed in a volume ratio of 5:40:20:0.5 and stirred (at 50° C., for 2 hours) to prepare a titanium sol solution. The Cu—Sr₄Al₁₄O₂₅:Eu²⁺,Dy³⁺-coated mesh slit and the titanium sol solution were put in a Teflon-liner container and subjected to hydrothermal synthesis in an autoclave (at 130° C., for 4 hours) to coat the surface of the Cu—Sr₄Al₁₄O₂₅:Eu²⁺,Dy³⁺-coated mesh slit with titanium dioxide to prepare a TiO₂/Cu—Sr₄Al₁₄O₂₅:Eu²⁺,Dy³⁺-coated mesh slit. The reactor was naturally cooled at room temperature and the TiO₂/Cu—Sr₄Al₁₄O₂₅:Eu²⁺,Dy³⁺-coated mesh slit was collected and dried (at 100° C., for 2 hours). Then, the coated titanium dioxide was heat-treated in an electric furnace (at 450° C., for 2 hours) for crystallization of the titanium dioxide into an anatase phase.

The prepared TiO₂/Cu—Sr₄Al₁₄O₂₅:Eu²⁺,Dy³⁺-coated mesh slits were stacked to manufacture a filter and rubber packing was inserted between the mesh slits to prevent contact therebetween (See FIG. 3A).

### (2) Comparative Example: Preparation of Filter for Air Purification 2

Sr₄Al₁₄O₂₅:Eu²⁺,Dy³⁺ powder, as a phosphor, and a mixture of Na₂SiO₃, as a binder, and zeolite powder were weighed in a weight ratio of 1:0.5 to 1 and then mixed with distilled water by stirring to prepare a slurry. The phosphor slurry was injected into a silicone mold having a diameter of about 3 mm and demolded to manufacture a semi-solid photoluminescent support in the form of particles in a large quantity. The semi-solid photoluminescent support was dried at room temperature (at about 25° C., for 6 hours) and in an electric oven (at 100° C., for 2 hours) and heat-treated in an electric furnace (at 350° C., for 2 hours) to obtain a Sr₄Al₁₄O₂₅:Eu²⁺,Dy³⁺ support in the form of hard light-emitting beads in a bulk form.

Subsequently, copper nitrate trihydrate was dissolved in ethanol and stirred at 70° C. for 1 hour to prepare a copper solution having a concentration of 0.62 M to 0.165 M. The Sr₄Al₁₄O₂₅:Eu²⁺,Dy³⁺ support prepared as described above was added to the copper solution, followed by sonication for 10 minutes. The resultant was dried at room temperature (at about 25° C., for 3 hours) and in an electric oven (at 100° C., for 2 hours) and heat-treated in an electric furnace (in a 90% N₂-10% H₂ reducing atmosphere, at 300° C., for 2 hours) to prepare a Cu—Sr₄Al₁₄O₂₅:Eu²⁺,Dy³⁺ support.

Subsequently, titanium tetra-isopropoxide (TTIP, Ti(OCH(CH₃)₂)₄) as a titanium precursor, hydrous ethanol, an acid solution (HNO₃), and distilled water were mixed in a volume ratio of 5:40:20:0.5 and stirred (at 50° C., for 2 hours) to prepare a titanium sol solution. The Cu—Sr₄Al₁₄O₂₅:Eu²⁺,Dy³⁺ support and the titanium sol solution were put in a Teflon-liner container and subjected to hydrothermal synthesis in an autoclave (at 130° C., for 4 hours) to coat the surface of the Cu—Sr₄Al₁₄O₂₅:Eu²⁺,Dy³⁺ support with titanium dioxide to prepare TiO₂/Cu—Sr₄Al₁₄O₂₅:Eu²⁺,Dy³⁺ beads. The reactor was naturally cooled at room temperature and the TiO₂/Cu—Sr₄Al₁₄O₂₅:Eu²⁺,Dy³⁺ beads were filtered and dried (at 100° C., for 2 hours). Then, the coated titanium dioxide was heat-treated in an electric furnace (at 450° C., for 2 hours) for crystallization of the titanium dioxide into an anatase phase to obtain TiO₂/Cu—Sr₄Al₁₄O₂₅:Eu²⁺,Dy³⁺ beads.

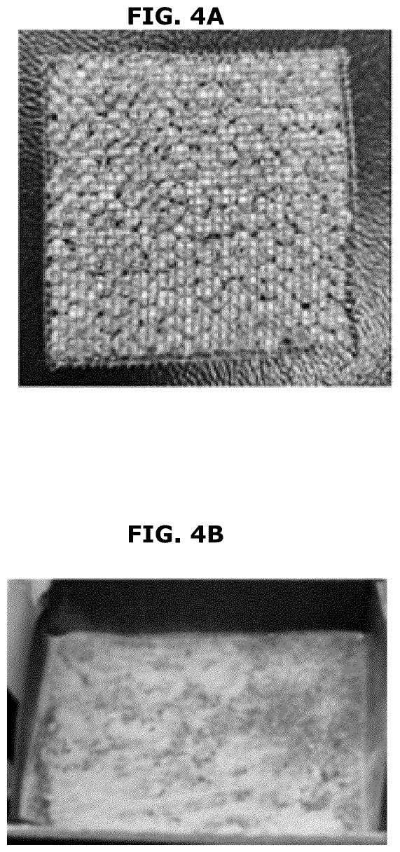

The prepared TiO₂/Cu—Sr₄Al₁₄O₂₅:Eu²⁺,Dy³⁺ beads were filled in the metal mesh frame to prepare a filter (See FIG. 4A).

### Experimental Example 1: Evaluation of Vibration Durability

In order to identify vibration durability of the filters prepared in the example and comparative example, the filters of the example and comparative example were applied to vehicles and driven 43000 km for 3.5 months.

FIGS. 3B and 4B are photographs of fine powder generated in a black box after a vehicle driving test using the filters prepared according to the example and comparative example for 3.5 months. Referring to FIGS. 3B and 4B, it may be confirmed that generation of fine powder was significantly decreased in the filter prepared according to the example compared to that prepared according to the comparative example. As such, it may be confirmed that a considerably large amount of fine powder is generated in a vibrating environment when the photocatalyst-beads are brought into contact with each other as shown in the case of the comparative example. The fine powder is generated by breakage or detachment of the photocatalyst coated on the beads and the like caused by friction therebetween.

### Experimental Example 2: Evaluation of Photocatalytic Oxidation Performance

To evaluate photocatalytic oxidation performance of the filters, a deodorization test was conducted using toluene, formaldehyde, and ammonia gas. In this regard, the filters of the example and comparative example were prepared in the same size of 4.0*4.0*0.8 cm (width*length*height) and exposed to a 455 nm LED array, as a light source, for 60 minutes. In this case, the deodorization test was conducted with a gas concentration of 30 ppm and a gas volume of 3 L of toluene/formaldehyde and 10 L of ammonia, and photocatalytic oxidation rates of the gases are shown in Table 1 below.

TABLE 1

| Type of gas | Example | Comparative Example |
|---|---|---|
| Toluene | 14.4% | 18.1% |
| Formaldehyde | 51.0% | 61.3% |
| Ammonia | 91.5% | 100% |

Referring to Table 1, in the case where the filters of the example and comparative example are prepared in the same size, since an amount of the phosphor and a specific surface area coated with TiO₂ of the example were decreased compared to the comparative example, performance of deodorization was slightly decrease. However, in the case of increasing the amount of the phosphor and the TiO₂ content by adjusting the filter size, the number of stacked mesh slits, photocatalytic oxidation efficiency is also expected to increase. In addition, the photocatalyst beads break in a vibrating environment in the case of the comparative example so that the amount of the photocatalyst decreases, as confirmed in Experimental Example 1. Thus, it is expected that photocatalytic oxidation performance thereof also deteriorate in the case of using the filter in an environment where vibrations continuously occur as in a vehicle.

According to the present disclosure, the filter for air purification according to the present disclosure may maximize photocatalytic oxidation efficiency by coating the metal mesh support with a light-emitting phosphor, supporting the transition metal particles capable of improving photocatalytic oxidation efficiency on the phosphor layer, and coating the photocatalyst material having photocatalytic oxidation function.

In addition, the filter for air purification according to the present disclosure may minimize breakage or detachment of coated materials by aligning the mesh supports coated with the phosphor, the transition metal, and the photocatalyst to be spaced apart from each other not to be in contact with each other.

In addition, problems of generation of fine powder and deterioration in photocatalytic oxidation performance may be prevented by minimizing breakage or detachment of coated materials.

However, the effects obtainable by the present disclosure are not limited to the aforementioned effects, and any other effects not mentioned herein will be clearly understood from the following description by those skilled in the art to which the present disclosure pertains.

Although embodiments of the disclosure have been shown and described, it would be appreciated by those having ordinary skill in the art that changes may be made in these embodiments without departing from the principles and spirit of the disclosure, the scope of which is defined in the claims and their equivalents.

1. A filter for air purification comprising a plurality of mesh slits stacked, wherein each of the plurality of mesh slits comprises:
a metal mesh support;
a phosphor layer coated on a surface of the mesh metal support;
a plurality of transition metal particles loaded on the phosphor layer; and
a photocatalyst layer coated on the phosphor layer on which the plurality of transition metal particles are loaded.

2. The filter for air purification according to claim 1, wherein a rubber packing is disposed between each of the plurality of mesh slits.

3. The filter for air purification according to claim 1, wherein the phosphor layer comprises a phosphor material, a binder, and zeolite.

4. The filter for air purification according to claim 2, wherein the phosphor material comprises at least one selected from CaAl₂O₄:(Eu,Nd)-based, SrAl₂O₄:(Eu,Dy)-based, Sr₄Al₁₄O₂₅:(Eu,Dy)-based, BaAl₂O₄:(Eu,Dy)-based, (Sr,Ba)₂MgSi₂O₇:(Eu,Dy)-based, Ba₄(Si₃O₈)₂:(Eu,Dy)-based, and [Ca,Sr,Ba]—Al—O-based compounds.

5. The filter for air purification according to claim 2, wherein the binder comprises at least one selected from sodium silicate (Na₂SiO₃), sodium polyphosphate (NaPO₃)ₙ, liquid silica, and glaze.

6. The filter for air purification according to claim 1, wherein the plurality of transition metal particles comprise at least one selected from Cr, Mn, Fe, Co, Ni, Cu, Zn, Zr, Ru, Rh, Pd, Ag, Ta, W, Pt, and Au.

7. The filter for air purification according to claim 1, wherein the photocatalyst layer comprises at least one photocatalyst material selected from titanium dioxide (TiO₂), graphite carbon nitride (g-C₃N₄), and a combination thereof.

8. The filter for air purification according to claim 1, wherein the photocatalyst layer further comprises SiO₂.

9. A method of manufacturing a filter for air purification, the method comprising:
preparing a plurality of metal mesh supports;
obtaining a phosphor-coated support by coating a surface of each of the plurality of metal mesh supports with a phosphor layer;
loading a plurality of transition metal particles on the phosphor-coated support;
coating a photocatalyst layer on the phosphor-coated support to obtain a plurality of mesh slits; and
stacking the plurality of mesh slits.

10. The method according to claim 9, wherein the obtaining of a phosphor-coated support by coating a surface of a support with a phosphor layer comprises:
preparing a phosphor slurry by mixing phosphor powder, a binder, zeolite, and distilled water; and
dipping the support in the phosphor slurry and drying and heat-treating a resultant.

11. The method according to claim 10, wherein the phosphor powder comprises at least one phosphor material selected from CaAl₂O₄:(Eu,Nd)-based, SrAl₂O₄:(Eu,Dy)-based, Sr₄Al₁₄O₂₅:(Eu,Dy)-based, BaAl₂O₄:(Eu,Dy)-based, (Sr,Ba)₂MgSi₂O₇:(Eu,Dy)-based, Ba₄(Si₃O₈)₂:(Eu,Dy)-based, and [Ca,Sr,Ba]—Al—O-based compounds; and
the binder comprises at least one selected from sodium silicate (Na₂SiO₃), sodium polyphosphate (NaPO₃)ₙ, liquid silica, and glaze.

12. The method according to claim 9, wherein loading the plurality of transition metal particles on the phosphor-coated support comprises:
preparing a transition metal support solution by dissolving a transition metal salt in an alcohol;
dipping the phosphor-coated support in the transition metal support solution, followed by sonication; and
drying and heat-treating a resultant.

13. The method according to claim 12, wherein the transition metal salt comprises at least one selected from copper nitrate trihydrate [Cu(NO₃)₂·3H₂O] and coper sulfate pentahydrate (CuSO₄·5H₂O).

14. The method according to claim 9, wherein the coating of the photocatalyst layer is performed by applying at least one method selected from a sol-gel method, a hydrothermal synthesis method, and a chemical vapor deposition (CVD) method thereto.

15. The method according to claim 9, wherein the coating of a photocatalyst layer on the phosphor-coated support on which the transition metal particles are loaded to obtain the plurality of mesh slits comprises:
preparing a photocatalyst sol by mixing a photocatalyst precursor, an alcohol-based solution, and an acid;
performing hydrothermal synthesis on the phosphor-coated support on which the transition metal particles are supported and the photocatalyst sol; and
drying and heat-treating a resultant.

16. The method according to claim 15, wherein the photocatalyst precursor comprises at least one selected from titanium tetra-isopropoxide [Ti(OCH(CH₃)₂)₄], tetrabutyl titanate [Ti(C₄H₉O)₄], and tetraethoxy titanium [Ti(OCH₂CH₃)₄].

17. The method according to claim 9, wherein the stacking of the plurality of mesh slits further comprises inserting a rubber packing between the plurality of mesh slits.
